(19) United States
(12) Patent Application Publication
(10) Pub. No.: US 2026/0249250 A1
(43) Pub. Date: Aug. 27, 2026

(54) POLYSILSESQUIOXANE/POLYETHERETHERKETONE COMPOSITE MEMBRANES AND METHODS FOR PREPARING THE SAME

(71) Applicant: CHANGZHOU UNIVERSITY, Changzhou (CN)

(72) Inventors: Meng GUO, Changzhou (CN); Jing ZHONG, Changzhou (CN); Xiuxiu REN, Changzhou (CN); Yang PAN, Changzhou (CN); Junzhu CHEN, Changzhou (CN); Rong XU, Changzhou (CN); Cheng XUE, Changzhou (CN)

(73) Assignee: CHANGZHOU UNIVERSITY, Changzhou (CN)

(21) Appl. No.: 19/530,433

(22) Filed: Feb. 5, 2026

Related U.S. Application Data
(63) Continuation-in-part of application No. PCT/CN2025/101181, filed on Jun. 16, 2025.

(30) Foreign Application Priority Data
Aug. 15, 2024 (CN) 202411118262.8

(51) Int. Cl. B01D 69/12; B01D 67/00; B01D 69/10; B01D 71/52; B01D 71/70

(52) U.S. Cl. CPC B01D 69/12; B01D 67/0083; B01D 67/0095; B01D 69/107; B01D 71/5222; B01D 71/70

(57) ABSTRACT

A polysilsesquioxane/polyetheretherketone composite membrane and a method for preparing the same are provided. The method includes: obtaining a polysilsesquioxane polymeric sol by mixing an ethanol solvent and a polysilsesquioxane precursor, sequentially adding deionized water and hydrochloric acid after complete dissolution, followed by constant-temperature water bath stirring for performing a polymerization reaction; and obtaining the polysilsesquioxane/polyetheretherketone composite membrane by coating the polysilsesquioxane polymeric sol on a polyetheretherketone support on which an intermediate layer is formed by knife coating.

### CROSS-REFERENCE TO RELATED APPLICATIONS

This application is a continuation-in-part application of International Application No. PCT/CN2025/101181, filed on Jun. 16, 2025, which claims priority to Chinese Patent Application No. 202411118262.8, filed on Aug. 15, 2024, the entire contents of each of which are hereby incorporated by reference.

### TECHNICAL FIELD

The present disclosure generally relates to a field of membrane separation technology, and in particular, to a polysilsesquioxane/polyetheretherketone (or polyether ether ketone, Peek) composite membrane and a method for preparing the same.

### BACKGROUND

Toluene is a widely used organic solvent in the petroleum and chemical industries (e.g., paints, rubber, glue, silicone sealants, inks, coatings, disinfectants, and leather tanning agents). However, during the industrial production process of toluene (e.g., the alkylation reaction of benzene and methanol), the product stream contains a mixture of methanol and toluene. Therefore, how to effectively separate methanol/toluene becomes very important. Dimethyl carbonate (DMC) is a green solvent integrating cleanliness and safety. DMC is a fuel additive and chemical raw material that can replace phosgene and other harmful substances. Since methanol (MeOH) can produce DMC, efficient separation of azeotropes is essential for the development of DMC. Recovery of organic solvents, including methanol, can provide immediate cost savings and significant environmental benefits for many chemical manufacturing companies. However, many organic-organic mixtures form azeotropic or near-boiling mixtures, which cannot always be easily recovered by conventional distillation. On this basis, it is crucial to develop an economical and effective separation technology for organic solvent separation.

Polyetheretherketone (or polyether ether ketone, Peek) consists of repeating phenylether and benzophenone units. Due to inherent structural characteristics, Peek exhibits strong chemical resistance in aggressive solvents. Furthermore, as an aromatic polymer, Peek is known for high operating temperatures and chemical resistance. The intrinsic properties of Peek are widely used in synthesizing various separation membranes, especially under harsh conditions. As an ultrafiltration membrane, Peek requires reduction of pore sizes on the membrane surface to perform pervaporation separation of organic mixed solvents.

In recent decades, extensive research has been conducted on various microporous materials, such as polymers, inorganic materials, and organic-inorganic hybrid materials, to obtain high-performance membranes for organic mixed solvent separation. Polysilsesquioxane hybrid membranes, formed by covalent bonding of organic bridging groups with two silicon atoms, dominate research on silicon hybrid materials for pervaporation (PV) and vapor permeation (VP) processes. This is mainly because the polysilsesquioxane network structure possesses remarkable thermal stability, chemical stability, adjustable pore sizes, affinity, and superior peroxide selectivity. These desirable properties originate from the uniform incorporation of different organic bridges into the polysilsesquioxane network via covalent bonds. Polysilsesquioxane has been proven to be a good precursor for preparing silicon-based organic-inorganic hybrid membranes. However, large-area deposition of defect-free polysilsesquioxane hybrid membranes on polymer substrates has always been a significant challenge.

All polysilsesquioxane hybrid membranes are prepared on inorganic supports (e.g., porous Al₂O₃ supports with pore sizes of about 200 nm-1000 nm). Costs of these ceramic membranes are always higher than those of polymer-based membranes. Therefore, polysilsesquioxane-Al₂O₃ membranes with complex preparation processes and high costs are not suitable for large-scale industrial utilization. To overcome this potential drawback, and to realize industrialization of membrane separation processes early, improve membrane preparation efficiency, and shorten membrane preparation time, the current challenge is large-area deposition of defect-free polysilsesquioxane hybrid membranes on existing polymer supports.

### SUMMARY

One or more embodiments of the present disclosure provide a method for preparing a polysilsesquioxane/polyetheretherketone (or polyether ether ketone, Peek) composite membrane. The method includes: obtaining a polysilsesquioxane polymeric sol by mixing an ethanol solvent and a polysilsesquioxane precursor, sequentially adding deionized water and hydrochloric acid after complete dissolution, followed by constant-temperature water bath stirring for performing a polymerization reaction; and obtaining the polysilsesquioxane/Peek composite membrane by coating the polysilsesquioxane polymeric sol on a Peek support on which an intermediate layer is formed by knife coating, wherein a molar ratio of the polysilsesquioxane precursor, the deionized water, and the hydrochloric acid is 1:(6-240):(0.2-1), a mass fraction of the polysilsesquioxane polymeric sol is 5 wt %-10 wt %; and the intermediate layer on the Peek support is formed by a process including: subjecting an aqueous phase piperazine solution and an organic phase 1,3,5-benzenetricarbonyl chloride solution to an interfacial polymerization reaction on the Peek support, or subjecting polydimethylsiloxane or amino silicone oil to an oxygen plasma modification on the Peek support.

In some embodiments, the polysilsesquioxane precursor includes one of 1,2-bis(triethoxysilyl)ethane (BTESE), 1,2-bis(triethoxysilyl)ethylene, 1,2-bis(triethoxysilyl)acetylene, 1,2-bis(triethoxyalkyl) methane, or 1,8-bis(triethoxyalkyl)octane.

In some embodiments, a stirring temperature of the constant-temperature water bath stirring is 30° C.-80° C., and a stirring time of the constant-temperature water bath stirring is 1 h-5 h.

In some embodiments, the subjecting the aqueous phase piperazine solution and the organic phase 1,3,5-benzenetricarbonyl chloride solution to the interfacial polymerization reaction on the Peek support includes: fixing the Peek support, pouring the aqueous phase piperazine solution onto a surface of the Peek support for residence and reaction, and removing an excess of the aqueous phase piperazine solution on the surface of the Peek support with a roller, then pouring the organic phase 1,3,5-benzenetricarbonyl chloride solution for residence to perform the interfacial polymerization reaction to form the intermediate layer, wherein a concentration of the aqueous phase piperazine solution is 0.5 wt %-2 wt %, a residence time of the aqueous phase piperazine solution is 1 min-5 min, a concentration of the organic phase 1,3,5-benzenetricarbonyl chloride solution is 0.01 wt %-0.1 wt %, and a residence time of the organic phase 1,3,5-benzenetricarbonyl chloride solution is 1 min-5 min; and a plasma treatment pressure of the oxygen plasma modification is 15 Pa-25 Pa, a treatment power of the oxygen plasma modification is 150 W-250 W, and a treatment time of the oxygen plasma modification is 60 s-180 s.

In some embodiments, the coating the polysilsesquioxane polymeric sol on the Peek support on which the intermediate layer is formed by the knife coating includes: fixing the Peek support on which the intermediate layer is formed, dropping the polysilsesquioxane polymeric sol onto one end of the Peek support, and coating a layer of the polysilsesquioxane polymeric sol with a knife at a constant speed, with a thickness of 200 nm-500 nm, and after calcination and drying, forming the polysilsesquioxane/Peek composite membrane.

In some embodiments, the constant speed for the coating is 0.5 cm/s-5 cm/s.

In some embodiments, a temperature of the calcination and drying is 50° C.-200° C., and a time of the calcination and drying is 15 min-60 min.

One or more embodiments of the present disclosure further provide a polysilsesquioxane/Peek composite membrane, which is prepared by the method above, wherein the polysilsesquioxane/Peek composite membrane includes a support, the intermediate layer, and a polymeric sol layer, wherein a material of the support is Peek, and a pore size of the support is 1 nm-100 nm, a material of the polymeric sol layer is the polysilsesquioxane polymeric sol, the intermediate layer is formed on the support, and the polymeric sol layer is coated on a surface of the intermediate layer.

One or more embodiments of the present disclosure further provide a method for separating an organic mixed solvent using the polysilsesquioxane/Peek composite membrane prepared by the method above. The method includes separating the organic mixed solvent by pervaporation using the polysilsesquioxane/Peek composite membrane, wherein a separation system of the organic mixed solvent includes methanol/toluene and methanol/dimethyl carbonate (DMC).

One or more embodiments of the present disclosure further provide a use of the polysilsesquioxane/Peek composite membrane in separating an organic mixed solvent. The use includes separating the organic mixed solvent by pervaporation using the polysilsesquioxane/Peek composite membrane, wherein a separation system of the organic mixed solvent includes methanol/toluene and methanol/DMC.

### BRIEF DESCRIPTION OF THE DRAWINGS

To more clearly illustrate the technical solutions in the embodiments of the present disclosure, the drawings used in the description of the embodiments are briefly introduced below. Obviously, the drawings in the following description are merely some embodiments of the present disclosure. For a person of ordinary skill in the art, other drawings may be obtained from these drawings without creative efforts.

FIG. 1 is a schematic diagram of a method for preparing a polysilsesquioxane/Polyetheretherketone (or polyether ether ketone, Peek) composite membrane in Example 1 of the present disclosure.

FIG. 2 is a schematic diagram of a membrane separation mechanism of the polysilsesquioxane/Peek composite membrane prepared in Example 1 of the present disclosure.

### DETAILED DESCRIPTION

To make the above objectives, features, and advantages of the embodiments of the present disclosure more comprehensible, the specific embodiments of the present disclosure are described in detail below with reference to the embodiments of the present disclosure.

Many specific details are set forth in the following description to facilitate a full understanding of the embodiments of the present disclosure. However, the embodiments of the present disclosure may also be implemented in other ways different from those described herein. Those skilled in the art can make similar extensions without departing from the spirit of the present disclosure. Therefore, the embodiments of the present disclosure are not limited by the specific embodiments disclosed below.

The term “an embodiment” or “embodiment” as used herein refers to a specific feature, structure, or characteristic that may be included in at least one implementation of the embodiments of the present disclosure. The phrase “in an embodiment” appearing in different places of the present disclosure does not necessarily refer to the same embodiment, nor is the phrase a separate or alternative embodiment mutually exclusive with other embodiments.

Generally, it is difficult to deposit a defect-free polysilsesquioxane hybrid membrane over a large area on a polymer substrate. The embodiments of the present disclosure provide a method for preparing a polysilsesquioxane/polyetheretherketone (or polyether ether ketone, Peek) composite membrane with a smooth and defect-free surface, which improves membrane preparation efficiency. The method includes forming an intermediate layer on a Peek support and then coating a polysilsesquioxane polymeric sol on the intermediate layer to obtain the polysilsesquioxane/Peek composite membrane. Moreover, the polysilsesquioxane/Peek composite membrane exhibits excellent performance in the separation and recovery of methanol/toluene and methanol/dimethyl carbonate (DMC).

The embodiments of the present disclosure provide the method for preparing the polysilsesquioxane/Peek composite membrane. In some embodiments, the method may be automatically performed by a control system. For example, the method may be performed through control instructions. The control system controls various components to complete steps of the method based on the control instructions. In some embodiments, the method may be semi-automatically performed. For example, one or more steps of the method may be manually performed by an operator. In some embodiments, the control system may include a processor. The processor may include a central processing unit, a microprocessor, an application-specific integrated circuit, etc., and is configured to execute the method to implement the technical solution provided by the embodiments of the present disclosure. The method includes: obtaining a polysilsesquioxane polymeric sol by mixing an ethanol solvent and a polysilsesquioxane precursor, sequentially adding deionized water and hydrochloric acid after complete dissolution, followed by constant-temperature water bath stirring for performing a polymerization reaction; and obtaining a polysilsesquioxane/Peek composite membrane by coating the polysilsesquioxane polymeric sol on a Peek support on which an intermediate layer is formed by knife coating.

In some embodiments, the polysilsesquioxane precursor includes one of 1,2-bis(triethoxysilyl)ethane (BTESE), 1,2-bis(triethoxysilyl)ethylene, 1,2-bis(triethoxysilyl)acetylene, 1,2-bis(triethoxyalkyl) methane, or 1,8-bis(triethoxyalkyl)octane.

In some embodiments, a mass fraction of the ethanol solvent is in a range of 30 wt %-95 wt %. In some embodiments, the mass fraction of the ethanol solvent is in a range of 33.57 wt %-93.38 wt %. In some embodiments, the mass fraction of the ethanol solvent is in a range of 40 wt %-90 wt %. In some embodiments, the mass fraction of the ethanol solvent is in a range of 50 wt %-80 wt %. In some embodiments, the mass fraction of the ethanol solvent is in a range of 60 wt %-70 wt %. In some embodiments, the mass fraction of the ethanol solvent is 30 wt %, 33.57 wt %, 40 wt %, 50 wt %, 60 wt %, 70 wt %, 80 wt %, 90 wt %, 93.38 wt %, 95 wt %, etc.

In some embodiments, a mass ratio of the ethanol solvent to the polysilsesquioxane precursor is in a range of 5 wt %-15 wt %. In some embodiments, the mass ratio of the ethanol solvent to the polysilsesquioxane precursor is in a range of 5.35 wt %-14.89 wt %. In some embodiments, the mass ratio of the ethanol solvent to the polysilsesquioxane precursor is in a range of 6 wt %-14 wt %. In some embodiments, the mass ratio of the ethanol solvent to the polysilsesquioxane precursor is in a range of 8 wt %-10 wt %. In some embodiments, the mass ratio of the ethanol solvent to the polysilsesquioxane precursor is 5 wt %, 5.35 wt %, 6 wt %, 7 wt %, 8 wt %, 9 wt %, 10 wt %, 11 wt %, 12 wt %, 13 wt %, 14 wt %, 14.89 wt %, 15%, etc.

In some embodiments, a mass fraction of the hydrochloric acid is in a range of 0.5 wt %-1 wt %. In some embodiments, the mass fraction of the hydrochloric acid is in a range of 0.6 wt %-0.8 wt %. In some embodiments, the mass fraction of the hydrochloric acid is 0.5 wt %, 0.6 wt %, 0.7 wt %, 0.8 wt %, 0.9 wt %, 1 wt % etc.

In some embodiments, a molar ratio of the polysilsesquioxane precursor, the deionized water, and the hydrochloric acid is 1:(6-240):(0.2-1). In some embodiments, the molar ratio of the polysilsesquioxane precursor, the deionized water, and the hydrochloric acid is 1:(20-240):(0.2-1). In some embodiments, the molar ratio of the polysilsesquioxane precursor, the deionized water, and the hydrochloric acid is 1:(40-240):(0.2-1). In some embodiments, the molar ratio of the polysilsesquioxane precursor, the deionized water, and the hydrochloric acid is 1:(60-240):(0.2-1). In some embodiments, the molar ratio of the polysilsesquioxane precursor, the deionized water, and the hydrochloric acid is 1:(80-240):(0.2-1). In some embodiments, the molar ratio of the polysilsesquioxane precursor, the deionized water, and the hydrochloric acid is 1:(100-240):(0.2-1). In some embodiments, the molar ratio of the polysilsesquioxane precursor, the deionized water, and the hydrochloric acid is 1:(150-240):(0.2-1). In some embodiments, the molar ratio of the polysilsesquioxane precursor, the deionized water, and the hydrochloric acid is 1:(200-240):(0.2-1). In some embodiments, the molar ratio of the polysilsesquioxane precursor, the deionized water, and the hydrochloric acid is 1:(6-60):(0.2-1). In some embodiments, the molar ratio of the polysilsesquioxane precursor, the deionized water, and the hydrochloric acid is 1:(6-240):(0.2-0.8). In some embodiments, the molar ratio of the polysilsesquioxane precursor, the deionized water, and the hydrochloric acid is 1:(6-240):(0.2-0.5). In some embodiments, the molar ratio of the polysilsesquioxane precursor, the deionized water, and the hydrochloric acid is 1:60:0.2, 1:60:0.5, 1:60:1, 1:6:0.2, 1:100:0.2, 1:200:0.2, 1:240:0.2, etc.

In some embodiments, a stirring temperature of the constant-temperature water bath stirring for performing the polymerization reaction is 30° C.-80° C. In some embodiments, the stirring temperature of the constant-temperature water bath stirring for performing the polymerization reaction is 40° C. to 80° C. In some embodiments, the stirring temperature of the constant-temperature water bath stirring for performing the polymerization reaction is 40° C. to 70° C. In some embodiments, the stirring temperature of the constant-temperature water bath stirring for performing the polymerization reaction is 40° C. to 60° C. In some embodiments, the stirring temperature of the constant-temperature water bath stirring for performing the polymerization reaction is 40° C. to 50° C. In some embodiments, the stirring temperature of the constant-temperature water bath stirring for performing the polymerization reaction is 30° C., 40° C., 50° C., 60° C., 70° C., 80° C., etc.

In some embodiments, a stirring time of the constant-temperature water bath stirring for performing the polymerization reaction is 1 h-5 h. In some embodiments, the stirring time of the constant-temperature water bath stirring for performing the polymerization reaction is 2 h to 5 h. In some embodiments, the stirring time of the constant-temperature water bath stirring for performing the polymerization reaction is 2 h to 4 h. In some embodiments, the stirring time of the constant-temperature water bath stirring for performing the polymerization reaction is 2 h to 3 h. In some embodiments, the stirring time of the constant-temperature water bath stirring for performing the polymerization reaction is 1 h, 2 h, 3 h, 4 h, 5 h, etc.

The polysilsesquioxane polymeric sol refers to a colloidal solution formed after a reaction (e.g., a hydrolysis-polymerization reaction) of the polysilsesquioxane precursor under acidic catalysis.

In some embodiments, a mass fraction of the polysilsesquioxane polymeric sol is 5 wt %-10 wt %. In some embodiments, the mass fraction of the polysilsesquioxane polymeric sol is 5 wt %-7.5 wt %. In some embodiments, the mass fraction of the polysilsesquioxane polymeric sol is 7.5 wt %-10 wt %. In some embodiments, the mass fraction of the polysilsesquioxane polymeric sol is 5 wt %, 7.5 wt %, 10 wt %, or the like.

In some embodiments, the coating the polysilsesquioxane polymeric sol on the Peek support on which the intermediate layer is formed by the knife coating includes: fixing the Peek support on which the intermediate layer is formed, dropping the polysilsesquioxane polymeric sol onto one end of the Peek support, and coating a layer of the polysilsesquioxane polymeric sol with a knife at a constant speed, with a thickness of 200 nm-500 nm, and after calcination and drying, forming the polysilsesquioxane/Peek composite membrane.

Coating the layer of the polysilsesquioxane polymeric sol with the knife may form a polysilsesquioxane polymeric sol layer on the intermediate layer of the Peek support. In some embodiments, a thickness of the polysilsesquioxane polymeric sol layer is 200 nm-500 nm. In some embodiments, the thickness of the polysilsesquioxane polymeric sol layer is 300 nm-500 nm. In some embodiments, the thickness of the polysilsesquioxane polymeric sol layer is 300 nm-400 nm. In some embodiments, the thickness of the polysilsesquioxane polymeric sol layer is 200 nm, 300 nm, 400 nm, 500 nm, etc.

In some embodiments, the constant speed for the coating is 0.5 cm/s-5 cm/s. In some embodiments, the constant speed for the coating is 1 cm/s-5 cm/s. In some embodiments, the constant speed for the coating is 2 cm/s-5 cm/s. In some embodiments, the constant speed for the coating is 2 cm/s-4 cm/s. In some embodiments, the constant speed for the coating is 2 cm/s-3 cm/s. In some embodiments, the constant speed for the coating is 0.5 cm/s, 1 cm/s, 2 cm/s, 3 cm/s, 4 cm/s, 5 cm/s, or the like.

In some embodiments, a temperature of the calcination and drying is 50° C.-200° C. In some embodiments, the temperature of the calcination and drying is 50° C.-150° C. In some embodiments, the temperature of the calcination and drying is 50° C.-120° C. In some embodiments, the temperature of the calcination and drying is 50° C.-100° C. In some embodiments, the temperature of the calcination and drying is 50° C.-80° C. In some embodiments, the temperature of the calcination and drying is 100° C.-200° C. In some embodiments, the temperature of the calcination and drying is 120° C.-200° C. In some embodiments, the temperature of the calcination and drying is 50° C., 100° C., 120° C., 150° C., 180° C., 200° C., or the like.

In some embodiments, a time of the calcination and drying is 15 min-60 min. In some embodiments, the time of the calcination and drying is 20 min-60 min. In some embodiments, the time of the calcination and drying is 25 min-50 min. In some embodiments, the time of the calcination and drying is 25 min-40 min. In some embodiments, the time of the calcination and drying is 25 min-30 min. In some embodiments, the time of the calcination and drying is 15 min, 25 min, 30 min, 40 min, 50 min, 60 min, or the like.

In some embodiments, after the polysilsesquioxane polymeric sol layer undergoes the calcination and drying, the polymeric sol layer is formed.

The intermediate layer refers to a middle layer of the polysilsesquioxane/Peek composite membrane. The intermediate layer is used to facilitate preparation of the polysilsesquioxane/Peek composite membrane and improve performance of the prepared polysilsesquioxane/Peek composite membrane. For example, the intermediate layer has a function of reducing a pore size of the Peek support.

In some embodiments, the intermediate layer on the Peek support is formed by a process. The process includes subjecting an aqueous phase piperazine solution and an organic phase 1,3,5-benzenetricarbonyl chloride solution to an interfacial polymerization reaction on the Peek support.

The interfacial polymerization reaction refers to a polymerization reaction that occurs at an interface between two immiscible solvents (e.g., an aqueous phase and an organic phase) by monomers or prepolymers having high reactivity.

In some embodiments, the subjecting the aqueous phase piperazine solution and the organic phase 1,3,5-benzenetricarbonyl chloride solution to the interfacial polymerization reaction on the Peek support includes fixing the Peek support, pouring the aqueous phase piperazine solution onto a surface of the Peek support for residence and reaction, and removing an excess of the aqueous phase piperazine solution on the surface of the Peek support with a roller, then pouring the organic phase 1,3,5-benzenetricarbonyl chloride solution for residence to perform the interfacial polymerization reaction to form the intermediate layer.

The aqueous phase piperazine solution refers to an aqueous solution of piperazine. In some embodiments, a concentration of the aqueous phase piperazine solution is 0.5 wt %-2 wt %. In some embodiments, the concentration of the aqueous phase piperazine solution is 1 wt %-2 wt %. In some embodiments, the concentration of the aqueous phase piperazine solution is 0.5 wt %-1 wt %. In some embodiments, the concentration of the aqueous phase piperazine solution is 0.5 wt %, 1 wt %, 1.5 wt %, 2 wt %, or the like.

A residence time of the aqueous phase piperazine solution refers to a time that the aqueous phase piperazine solution stays on the surface of the Peek support before the excess of the aqueous phase piperazine solution is removed with the roller. During a process of pouring the aqueous phase piperazine solution onto the surface of the Peek support for the residence and reaction, the aqueous phase piperazine solution may infiltrate and be physically adsorbed on the Peek support.

In some embodiments, the residence time of the aqueous phase piperazine solution is 1 min-5 min. In some embodiments, the residence time of the aqueous phase piperazine solution is 2 min-5 min. In some embodiments, the residence time of the aqueous phase piperazine solution is 3 min-5 min. In some embodiments, the residence time of the aqueous phase piperazine solution is 3 min-4 min. In some embodiments, the residence time of the aqueous phase piperazine solution is 1 min, 2 min, 3 min, 4 min, 5 min, or the like.

The organic phase 1,3,5-benzenetricarbonyl chloride solution refers to a solution formed by dissolving 1,3,5-benzenetricarbonyl chloride in an organic solvent. In some embodiments, the organic solvent may include, but is not limited to, n-hexane, cyclohexane, heptane, or the like.

In some embodiments, a concentration of the organic phase 1,3,5-benzenetricarbonyl chloride solution is 0.01 wt %-0.1 wt %. In some embodiments, the concentration of the organic phase 1,3,5-benzenetricarbonyl chloride solution is 0.02 wt %-0.1 wt %. In some embodiments, the concentration of the organic phase 1,3,5-benzenetricarbonyl chloride solution is 0.05 wt %-0.1 wt %. In some embodiments, the concentration of the organic phase 1,3,5-benzenetricarbonyl chloride solution is 0.05 wt %-0.08 wt %. In some embodiments, the concentration of the organic phase 1,3,5-benzenetricarbonyl chloride solution is 0.01 wt %-0.05 wt %. In some embodiments, the concentration of the organic phase 1,3,5-benzenetricarbonyl chloride solution is 0.03 wt %-0.05 wt %. In some embodiments, the concentration of the organic phase 1,3,5-benzenetricarbonyl chloride solution is 0.01 wt %, 0.03 wt %, 0.05 wt %, 0.08 wt %, 0.1 wt %, or the like.

A residence time of the organic phase 1,3,5-benzenetricarbonyl chloride solution refers to a time of the interfacial polymerization reaction. In some embodiments, the residence time of the organic phase 1,3,5-benzenetricarbonyl chloride solution is 1 min-5 min. In some embodiments, the residence time of the organic phase 1,3,5-benzenetricarbonyl chloride solution is 1 min-3 min. In some embodiments, the residence time of the organic phase 1,3,5-benzenetricarbonyl chloride solution is 3 min-5 min. In some embodiments, the residence time of the organic phase 1,3,5-benzenetricarbonyl chloride solution is 1 min, 2 min, 3 min, 4 min, 5 min, or the like.

In some embodiments, after removing the excess of the aqueous phase piperazine solution on the surface of the Peek support with the roller and before pouring the organic phase 1,3,5-benzenetricarbonyl chloride solution, the method further includes acquiring surface image data of the Peek support; extracting a reflective feature and a texture feature of the surface of the Peek support according to the surface image data; and determining the concentration and the residence time of the organic phase 1,3,5-benzenetricarbonyl chloride solution according to the reflective feature and the texture feature.

The surface image data refers to image information reflecting the surface of the Peek support. The surface image data may include color, gloss, gloss uniformity, surface texture, visible defects, or the like.

The surface image data may be obtained by an image acquisition device (e.g., a visible light camera) capturing the surface of the Peek support. In some embodiments, the surface image data is a red green blue (RGB) image.

In some embodiments, extracting the reflective feature of the surface of the Peek support according to the surface image data includes performing image grayscale processing on the surface image data to obtain a single-channel grayscale image; setting pixel points with a grayscale greater than a preset brightness threshold in the single-channel grayscale image as a reflective region; and determining the reflective feature of the surface of the Peek support based on the reflective region.

In some embodiments, the preset brightness threshold is preset by a technician based on experience.

The reflective feature refers to a feature for characterizing a light reflection condition of the surface of the Peek support. For example, the reflective feature may include a reflective intensity, a reflective area, or the like.

In some embodiments, an average grayscale of a plurality of the pixel points in a plurality of the reflective regions may be used as the reflective intensity.

In some embodiments, a ratio of a total number of the plurality of pixel points in the plurality of reflective regions to a total number of pixel points in the single-channel grayscale image may be used as the reflective area.

The texture feature refers to a feature for characterizing a surface texture of the Peek support. For example, the texture feature may include a texture clarity.

The texture clarity refers to a metric for characterizing a distinguishable degree of the surface texture of the Peek support. For example, the texture clarity may be represented as any numerical value in (1, 10000). A larger numerical value of the texture clarity indicates a clearer surface texture of the Peek support.

In some embodiments, extracting the texture feature of the surface of the Peek support according to the surface image data includes: performing the image grayscale processing on the surface image data to obtain the single-channel grayscale image; calculating the texture clarity of the single-channel grayscale image based on a Laplacian variance manner, and using the texture clarity as the texture feature.

According to the reflective feature and the texture feature, the concentration and the residence time of the organic phase 1,3,5-benzenetricarbonyl chloride solution may be determined in a plurality of ways.

In some embodiments, according to the reflective feature and the texture feature, the concentration and the residence time of the organic phase 1,3,5-benzenetricarbonyl chloride solution may be determined through a preset rule. The preset rule may include that the concentration and the residence time of the organic phase 1,3,5-benzenetricarbonyl chloride solution are positively correlated with the reflective intensity and the reflective area and are negatively correlated with the texture clarity. The preset rule may be preset by the technician based on experience. For example, when the reflective intensity of the surface of the Peek support is strong, the reflective area is large, and the texture clarity is low, it indicates that the aqueous phase piperazine solution floats on the surface of the Peek support and does not penetrate into pores of the Peek support. The concentration and the residence time of the organic phase 1,3,5-benzenetricarbonyl chloride solution may be increased by a first preset percentage and a second preset percentage, respectively, to make the interfacial polymerization reaction more sufficient. In some embodiments, the first preset percentage and the second preset percentage may be preset by the technician based on experience.

In some embodiments, determining the concentration and the residence time of the organic phase 1,3,5-benzenetricarbonyl chloride solution according to the reflective feature and the texture feature may include: constructing a feature vector according to the reflective feature and the texture feature; and determining the concentration and the residence time of the organic phase 1,3,5-benzenetricarbonyl chloride solution by retrieving in a vector database based on the feature vector.

In some embodiments, a processor may concatenate the reflective feature and the texture feature to construct the feature vector.

The vector database refers to a database for determining the concentration and the residence time of the organic phase 1,3,5-benzenetricarbonyl chloride solution. In some embodiments, the vector database includes a plurality of reference vectors, and a reference concentration and a reference residence time of a reference organic phase 1,3,5-benzenetricarbonyl chloride solution corresponding to each reference vector.

In some embodiments, the processor may construct the reference vectors based on historical data (e.g., historical reflective features and historical texture features of surfaces of historical Peek supports after removing excesses of historical aqueous phase piperazine solutions from the surfaces of the historical Peek supports with the rollers and before pouring historical organic phase 1,3,5-benzenetricarbonyl chloride solutions).

In some embodiments, the processor may obtain a historical concentration and a historical residence time of a historical organic phase 1,3,5-benzenetricarbonyl chloride solution used in a historical interfacial polymerization reaction that produced a historical polysilsesquioxane/Peek composite membrane with the best quality, and use the historical concentration and the historical residence time as the reference concentration and the reference residence time of the reference organic phase 1,3,5-benzenetricarbonyl chloride solution.

The historical polysilsesquioxane/Peek composite membrane with the best quality refers to a historical polysilsesquioxane/Peek composite membrane with the smallest difference between a separation factor and a reference value, a largest permeation flux, and a lowest performance decay rate. The separation factor, the permeation flux, and the performance decay rate may be obtained by performing a pervaporation experiment on the historical polysilsesquioxane/Peek composite membrane. The reference value may be preset by the technician based on experience.

In some embodiments, the processor may determine a concentration and a residence time of an organic phase 1,3,5-benzenetricarbonyl chloride solution corresponding to the feature vector by respectively calculating a similarity between the reference vector and the feature vector. Merely by way of example, the processor may use a reference vector having a greatest similarity with the feature vector as a target vector, and use a reference concentration and a reference residence time of a reference organic phase 1,3,5-benzenetricarbonyl chloride solution corresponding to the target vector as the concentration and the residence time of the organic phase 1,3,5-benzenetricarbonyl chloride solution corresponding to the feature vector.

The similarity between the reference vector and the feature vector may be negatively correlated with a vector distance between the reference vector and the feature vector. The vector distance may be determined based on a cosine distance, or the like. Merely by way of example, the similarity may be a reciprocal of the vector distance.

In embodiments of the present disclosure, after removing the excess of the aqueous phase piperazine solution from the surface of the Peek support with the roller and before pouring the organic phase 1,3,5-benzenetricarbonyl chloride solution, determining the concentration and the residence time of the organic phase 1,3,5-benzenetricarbonyl chloride solution according to different surface conditions (e.g., the surface image data) of the Peek support can allow the interfacial polymerization reaction between the aqueous phase piperazine solution and the organic phase 1,3,5-benzenetricarbonyl chloride solution to proceed more fully, which can further allow the prepared intermediate layer to better regulate the pore size of the Peek support to resist swelling of the Peek support by toluene or the like in the organic mixed solvent, and can further improve a service life of the prepared polysilsesquioxane/Peek composite membrane.

In some embodiments, during the interfacial polymerization reaction, the method further includes: at every preset period, constructing a historical light scattering curve according to historical light scattering signals; determining reaction rates at a plurality of historical time points according to the historical light scattering curve; and adjusting the concentration and the residence time of the organic phase 1,3,5-benzenetricarbonyl chloride solution according to the reaction rates.

The preset period may be preset by the technician based on experience. Merely by way of example, the preset period includes 10 s, 20 s, etc.

The historical light scattering signals refer to signals corresponding to a plurality of historical time points within a previous preset period, and the signals reflect a degree of light scattering by a membrane layer generated during the interfacial polymerization reaction. Merely by way of example, the historical light scattering signals include historical light scattering intensities at the plurality of historical time points within the previous preset period.

In some embodiments, the historical light scattering signal may be obtained by monitoring the membrane layer generated during the interfacial polymerization reaction with an optical sensor. The optical sensor may include a lateral laser scattering sensor or a fiber optic turbidimeter.

The historical light scattering curve refers to a change curve reflecting a progress of the interfacial polymerization reaction and a growth trend of the membrane layer.

An abscissa of the historical light scattering curve may be the plurality of historical time points within the previous preset period. An ordinate of the historical light scattering curve may be the historical light scattering intensity. The historical light scattering intensities correspond to the plurality of historical time points within the previous preset period.

In some embodiments, the constructing the historical light scattering curve according to the historical light scattering signals includes performing filtering processing on the historical light scattering signals to obtain the historical light scattering curve.

The reaction rate refers to a rate of the interfacial polymerization reaction.

In some embodiments, the determining the reaction rates at the plurality of historical time points according to the historical light scattering curve includes: calculating an instantaneous slope of the historical light scattering curve at each historical time point, and using the instantaneous slope as the reaction rate (which may also be referred to as an instantaneous reaction rate) at the each historical time point.

In some embodiments, the adjusting the concentration and the residence time of the organic phase 1,3,5-benzenetricarbonyl chloride solution according to the reaction rates includes: determining an average reaction rate of the previous preset period according to the instantaneous reaction rates; comparing the average reaction rate with a preset rate threshold; and reducing the concentration and the residence time of the organic phase 1,3,5-benzenetricarbonyl chloride solution in response to a determination that the average reaction rate is greater than the preset rate threshold. Merely by way of example, the residence time of the organic phase 1,3,5-benzenetricarbonyl chloride solution may be reduced by a third preset percentage in response to the determination that the average reaction rate is greater than the preset rate threshold. As another example, the concentration of the organic phase 1,3,5-benzenetricarbonyl chloride solution may be diluted in real time in response to the determination that the average reaction rate is greater than the preset rate threshold. Merely by way of example, the concentration of the organic phase 1,3,5-benzenetricarbonyl chloride solution may be diluted by injecting the organic solvent of the organic phase 1,3,5-benzenetricarbonyl chloride solution in real time. As another example, the concentration of the organic phase 1,3,5-benzenetricarbonyl chloride solution may be reduced by a fourth preset percentage in a next preset period in response to the determination that the average reaction rate is greater than the preset rate threshold. The preset rate threshold, the third preset percentage, and the fourth preset percentage may be preset by the technician based on experience. Merely by way of example, the third preset percentage and the fourth preset percentage may be 60%, 70%, 80%, etc.

In embodiments of the present disclosure, the interfacial polymerization reaction is regulated by adjusting the concentration and the residence time of the organic phase 1,3,5-benzenetricarbonyl chloride solution, which can produce a flat and ultrathin intermediate layer. The intermediate layer can reduce pore sizes of the Peek support and support the polymeric sol layer, further enabling the prepared polysilsesquioxane/Peek composite membrane to achieve precise sieving of methanol molecules, and to have a large permeation flux while ensuring a high separation factor.

In some embodiments, the intermediate layer on the Peek support is formed by a process. The process includes subjecting polydimethylsiloxane (PDMS) or amino silicone oil to an oxygen plasma modification on the Peek support.

In some embodiments, a plasma treatment pressure of the oxygen plasma modification for PDMS or the amino silicone oil on the Peek support is 15 Pa-25 Pa. In some embodiments, the plasma treatment pressure of the oxygen plasma modification is 15 Pa to 25 Pa. In some embodiments, the plasma treatment pressure of the oxygen plasma modification is 15 Pa to 20 Pa. In some embodiments, the plasma treatment pressure of the oxygen plasma modification is 20 Pa to 25 Pa. In some embodiments, the plasma treatment pressure of the oxygen plasma modification is 15 Pa, 20 Pa, 25 Pa, etc.

In some embodiments, a treatment power of the oxygen plasma modification is 150 W-250 W. In some embodiments, the treatment power of the oxygen plasma modification is 180 W to 250 W. In some embodiments, the treatment power of the oxygen plasma modification is 200 W to 250 W. In some embodiments, the treatment power of the oxygen plasma modification is 150 W, 200 W, 250 W, etc.

In some embodiments, a treatment time of the oxygen plasma modification is 60 s-180 s. In some embodiments, the treatment time of the oxygen plasma modification is 100 s to 180 s. In some embodiments, the treatment time of the oxygen plasma modification is 120 s to 180 s. In some embodiments, the treatment time of the oxygen plasma modification is 60 s to 120 s. In some embodiments, the treatment time of the oxygen plasma modification is 120 s to 150 s. In some embodiments, the treatment time of the oxygen plasma modification is 120 s to 180 s. In some embodiments, the treatment time of the oxygen plasma modification is 60 s, 100 s, 120 s, 150 s, 180 s, etc.

In embodiments of the present disclosure, the polysilsesquioxane/Peek composite membrane with a flat and defect-free surface is prepared by forming the intermediate layer on the Peek support and then coating the polysilsesquioxane polymeric sol on the intermediate layer using the knife coating. The preparation method not only optimizes a sol-gel preparation process, improves membrane preparation efficiency, but also reduces membrane preparation costs by using a lower-cost Peek support as a substrate.

The present disclosure also provides a polysilsesquioxane/Peek composite membrane. The polysilsesquioxane/Peek composite membrane is prepared by the above-described preparation method. The polysilsesquioxane/Peek composite membrane includes a support, the intermediate layer, and a polymeric sol layer.

The support refers to a base layer of the polysilsesquioxane/Peek composite membrane, which provides mechanical support. In some embodiments, a material of the support is Peek.

In some embodiments, a pore size of the support is 1 nm-100 nm. In some embodiments, the pore size of the support is 10 nm-100 nm. In some embodiments, the pore size of the support is 20 nm-100 nm. In some embodiments, the pore size of the support is 50 nm-100 nm. In some embodiments, the pore size of the support is 80 nm-100 nm. In some embodiments, the pore size of the support is 1 nm-50 nm. In some embodiments, the pore size of the support is 10 nm-50 nm. In some embodiments, the pore size of the support is 30 nm-50 nm. In some embodiments, the pore size of the support is 50 nm-80 nm. In some embodiments, the pore size of the support is 1 nm, 20 nm, 50 nm, 80 nm, 100 nm, etc.

In some embodiments, the intermediate layer is formed on the support.

In some embodiments, a material of the polymeric sol layer is the polysilsesquioxane polymeric sol. In some embodiments, the polymeric sol layer is coated on a surface of the intermediate layer. That is, the polymeric sol layer is located on the intermediate layer.

More descriptions regarding the intermediate layer and the polymeric sol layer may be found in the foregoing description of the present disclosure.

The present disclosure also provides a method for separating an organic mixed solvent using the polysilsesquioxane/Peek composite membrane prepared by the above-described preparation method. The method includes separating the organic mixed solvent by pervaporation using the polysilsesquioxane/Peek composite membrane.

In some embodiments, a separation system of the organic mixed solvent includes methanol/toluene and methanol/DMC.

The polysilsesquioxane/Peek composite membrane of the present disclosure has an excellent effect for separation and recovery of methanol/toluene and methanol/DMC.

Raw materials used in the embodiments of the present disclosure are commercially available ordinary materials in the art unless otherwise specified. A pore size of the used Peek support is 50 nm.

### Example 1

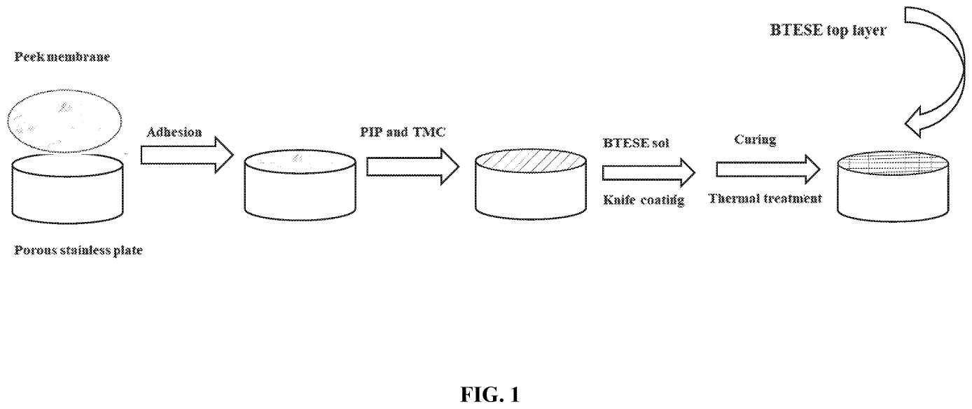

FIG. 1 is a schematic diagram of a method for preparing a polysilsesquioxane/Peek composite membrane in Example 1 of the present disclosure.

Referring to the schematic diagram in FIG. 1, a method for preparing the composite membrane using Peek as a support is provided in EXAMPLE 1. A structural formula of a BTESE precursor used is shown in Formula (I):

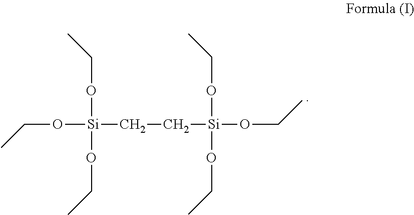 [SMILES: CCO[Si](CC[Si](OCC)(OCC)OCC)(OCC)OCC]

The method includes the following specific steps.

### 1) Preparation of a Composite Silicon Polymeric Sol (Also Referred to as a Polysilsesquioxane Polymeric Sol).

A molar ratio of BTESE, deionized water, and hydrochloric acid is 1:60:0.2.

5.93 g of an ethanol solvent and 3.05 g of the deionized water were mixed. 0.02 g of the hydrochloric acid and 1 g of a polysilsesquioxane precursor (i.e., BTESE) were added sequentially after complete dissolution. Constant-temperature water bath stirring was performed at 40° C. for 2 h for a polymerization reaction to obtain a composite silicon polymeric sol (also referred to as a BTESE sol) with a mass fraction of 10 wt %.

### 2) Preparation of an Intermediate Layer

1 g of anhydrous piperazine (PIP) and 99 g of deionized water were mixed and stirred at room temperature until completely dissolved to obtain an aqueous phase piperazine solution with a mass fraction of 1 wt %.

0.01 g of 1,3,5-benzenetricarbonyl chloride (TMC) and 20 g of n-hexane were mixed and stirred at room temperature until completely dissolved to obtain an organic phase 1,3,5-benzenetricarbonyl chloride solution with a mass fraction of 0.05 wt %.

The Peek support was fixed on a membrane module (a conventional container for fixing a membrane) for interfacial polymerization reactions. The aqueous phase piperazine solution (1 wt %) was poured into the membrane module with a residence time of 3 min. Then, a roller was used to remove an excess of the aqueous phase piperazine solution on a surface of the Peek support. Subsequently, the organic phase 1,3,5-benzenetricarbonyl chloride solution (0.05 wt %) was poured into the membrane module with a residence time of 5 min. An interfacial polymerization reaction was thereby completed to form the intermediate layer.

### 3) Preparation of the Polysilsesquioxane/PEEK Composite Membrane

The polysilsesquioxane polymeric sol was coated on the surface of the Peek support on which the intermediate layer is formed by knife coating. Specifically, the Peek support (also referred to as a Peek membrane) was fixed on a porous stainless plate with adhesive tape. 200 μL of the BTESE sol (10 wt %) was dropped onto one end of the Peek support. Then, a knife was used to coat a layer of the BTESE sol at a constant speed of 2 cm/s to obtain a BTESE sol layer with a thickness of about 300 nm. The coated Peek support was calcined and dried (also referred to as a thermal treatment and curing) at 120° C. for 25 min to obtain the polysilsesquioxane/Peek composite membrane using the Peek as the support.

### Example 2

This example differs from Example 1 in that the intermediate layer in the preparation process was formed by subjecting PDMS to an oxygen plasma modification. A plasma treatment pressure was set to 20 Pa, a treatment power was set to 200 W, and a treatment time was set to 120 s. Other process parameters were the same as those in Example 1, and a polysilsesquioxane/Peek composite membrane of this example was obtained.

### Example 3

This example differs from Example 1 in that the intermediate layer in the preparation process was formed by subjecting amino silicone oil to an oxygen plasma modification. The plasma treatment pressure was set to 20 Pa, the treatment power was set to 200 W, and the treatment time was set to 120 s. Other process parameters were the same as those in Example 1, and a polysilsesquioxane/Peek composite membrane of this example was obtained.

### Comparative Example 1

This comparative example differs from Example 1 in that a step of preparing the intermediate layer was omitted, i.e., step 2) was omitted. Other steps were performed with reference to Example 1. A polysilsesquioxane/Peek composite membrane of this comparative example was obtained.

### Comparative Example 2

This comparative example differs from Example 1 in that a temperature of calcination and drying in step 3) of the preparation process was adjusted to 50° C. Other process parameters were the same as those in Example 1. A polysilsesquioxane/Peek composite membrane of this comparative example was obtained.

### Separation Test

Membranes prepared in Examples 1-3 and Comparative Examples 1 and 2 were used for pervaporation demethanolization of a methanol/toluene solution and a methanol/DMC solution. A feed pressure was controlled at 100 KPa, and a test temperature was 50° C. The methanol/toluene solution has a composition of 10 wt % methanol and 90 wt % toluene. The methanol/DMC solution has a composition of 10 wt % methanol and 90 wt % DMC. A methanol flux and a separation factor are determined and the results are shown in Table 1.

TABLE 1

| 10 wt % methanol/toluene solution | | | 10 wt % methanol/DMC solution | |
|---|---|---|---|---|
| | methanol | | methanol | |
| | flux | separation | flux | separation |
| | [g h⁻¹ m⁻²] | factor | [g h⁻¹ m⁻²] | factor |
| Example 1 | 1650 | 83 | 830 | 24 |
| Example 2 | 1035 | 14 | 753 | 5 |
| Example 3 | 1126 | 9 | 787 | 2 |
| Compar- | 8632 | 1 | 9426 | 1 |
| ative | | | | |
| Example 1 | | | | |
| Compar- | 1997 | 11 | 1530 | 3 |
| ative | | | | |
| Example 2 | | | | |

As can be seen from Table 1, the change of the intermediate layer also significantly affects the separation effect of the membrane. The oxygen plasma modification of PDMS can activate the surface of PDMS, enhance hydrophilicity of PDMS, enhance interfacial interaction, and facilitate diffusion of monolayer molecules to the surface of PDMS, causing that the PDMS matrix surface is modified. Finally, methanol molecules can permeate the membrane in large quantities, and the methanol permeation flux increases. By performing the oxygen plasma modification of the amino silicone oil, the amine group in the amino silicone oil can react with BTESE, enhance hydrophilicity of the membrane, and promote permeation of a large amount of methanol through the membrane. An ultrathin polymeric layer at a nanometer scale (which may be referred to as the intermediate layer) can be prepared through the interfacial polymerization reaction. The pore size and the thickness of the intermediate layer can be adjusted by changing the residence time of the organic phase, thereby making the size of the membrane better controlled, which is beneficial for reducing the pore size of the membrane and enabling the BTESE top layer to exert better separation performance.

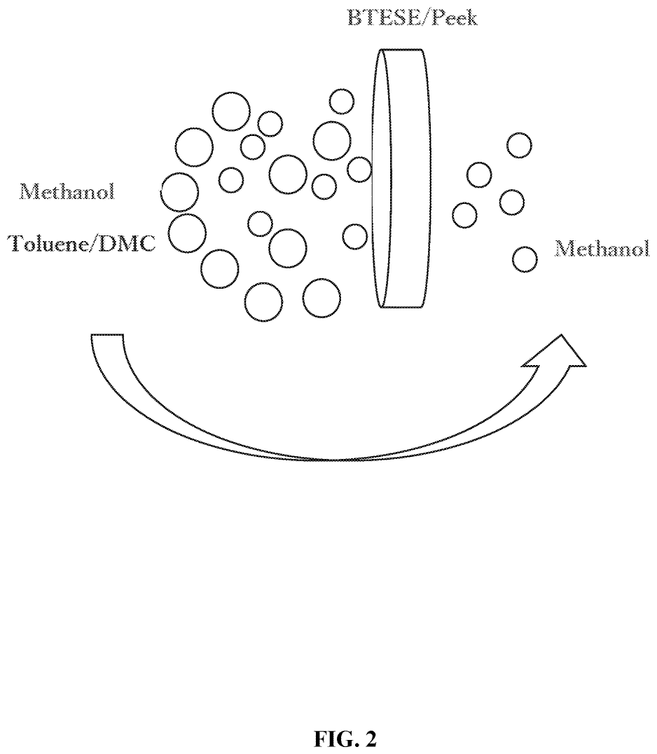

FIG. 2 is a schematic diagram of a membrane separation mechanism of the polysilsesquioxane/Peek composite membrane prepared in Example 1 of the present disclosure.

As can be seen from FIG. 2, due to adjustment of the pore size, methanol molecules with a smaller molecular size can permeate the membrane, while toluene or DMC molecules with a larger molecular size cannot permeate the membrane.

After the polysilsesquioxane polymeric sol is coated on the Peek support, different temperatures of the calcination and drying have a certain influence on the membrane separation performance. Low-temperature calcination and drying increases the content of Si—OH groups on the membrane surface, increases the pore size, and makes methanol molecules more likely to pass through the membrane, thereby increasing the methanol permeation flux.

Modification of the surface of the Peek support with the intermediate layer can achieve the purpose of reducing the pore size of the surface of the Peek support. For modification of the intermediate layer, three different modification manners are adopted. The first manner is performing the interfacial polymerization reaction between the aqueous phase piperazine solution and the organic phase 1,3,5-benzenetricarbonyl chloride solution on the surface of the Peek support. The second manner is performing the oxygen plasma modification of PDMS on the surface of the Peek support to react with oxygen ions. The third manner is performing the oxygen plasma modification of the amino silicone oil on the surface of the Peek support. The intermediate layer is formed by the above three manners to reduce the surface pores of the Peek support.

In the interfacial polymerization reaction, two monomers or prepolymers with high reactivity (usually polyamines and polyacyl chlorides) are used to undergo a polymerization reaction at an interface of two immiscible solvents (i.e., an aqueous phase and an organic phase), thereby forming an ultrathin layer on a porous support. The most common interfacial polymerization reaction for preparing a composite membrane is a reaction between an aqueous phase piperazine solution and an organic phase 1,3,5-benzenetricarbonyl chloride solution. Because an ultrathin polymeric layer at a nanometer scale can be prepared through the interfacial polymerization reaction, such membranes have both high selectivity and high permeability under low pressure and thus have attracted much attention and importance in the field of membrane separation.

### Example 4

This example differs from Example 1 in that the residence time of the organic phase 1,3,5-benzenetricarbonyl chloride solution in the membrane module in step 3) of Example 1 was adjusted to 1 min, 3 min, and 7 min, respectively. The remaining process parameters were the same as those in Example 1. Different polysilsesquioxane/Peek composite membranes of this example were obtained. The membrane performance was tested with reference to the above separation test manner. Compared with Example 1, the results are shown in Table 2.

TABLE 2

| 10 wt % methanol/toluene solution | | | 10 wt % methanol/DMC solution | |
|---|---|---|---|---|
| | methanol flux | separation | methanol flux | separation |
| | [g h⁻¹ m⁻²] | factor | [g h⁻¹ m⁻²] | factor |
| 5 min | 1650 | 83 | 830 | 24 |
| 1 min | 1983 | 7 | 927 | 3 |
| 3 min | 1764 | 21 | 891 | 15 |
| 7 min | 1372 | 64 | 690 | 22 |

As can be seen from TABLE 2, the residence time of the organic phase is crucial to the pore size of the intermediate layer of the membrane. At a residence time of 5 min, the separation factor is the largest. This indicates that the intermediate layer is completely formed and fills the surface pores of the Peek support, allowing the top layer to exhibit better separation performance. When the residence time is 1 min and 3 min, although the separation performance exists, the separation factor is too low. This indicates that the residence time is too short, resulting in an intermediate layer that cannot completely fill the surface pores of the Peek support.

### Example 5

This example differs from Example 1 in that the BTESE concentration in the preparation process was adjusted to 0, 5 wt %, 7.5 wt %, and 12.5 wt %, respectively. The remaining process parameters were the same as in Example 1. Polysilsesquioxane/Peek composite membranes of this example were obtained. The membrane performance was tested according to the separation test manner described above. Compared with Example 1, the results are shown in TABLE 3.

TABLE 3

| 10 wt % methanol/toluene solution | | | | 10 wt % methanol/DMC solution | |
|---|---|---|---|---|---|
| | | methanol flux | separation | methanol flux | separation |
| concentration | | [g h⁻1 m⁻²] | factor | [g h⁻¹ m⁻²] | factor |
| 12.5 | wt % | 1398 | 56 | 439 | 17 |
| 10 | wt % | 1650 | 83 | 830 | 24 |
| 7.5 | wt % | 1923 | 22 | 1340 | 5 |
| 5 | wt % | 1354 | 5 | 700 | 3 |

| | | | | |
|---|---|---|---|---|
| 0 | 12500 | 1 | 9790 | 1 |

As can be seen from Table 3, for both the methanol/toluene separation system and the methanol/DMC separation system, the membrane prepared in Example 1 of the present disclosure has the best separation selectivity. When the concentration of the BTESE sol in the membrane preparation process is changed to 7.5 wt %, the methanol flux increases slightly, but the selectivity decreases significantly. When the concentration of the BTESE sol is changed to 5 wt %, both the methanol flux and the separation factor decrease significantly. This is because the reduced concentration of the BTESE sol cannot completely fill the surface pores of the Peek support having the intermediate layer, resulting in defects in the membrane. Therefore, both the flux and the separation factor decrease.

Embodiments of the present disclosure provide the method for preparing the polysilsesquioxane/Peek composite membrane for separation of the organic mixed solvent by pervaporation. To address the problem of depositing a large-area and defect-free hybrid silica membrane on a polymer substrate, a new membrane preparation method is proposed from the perspectives of simplicity of the membrane preparation process, industrialization of membrane separation technology, and improvement of membrane preparation efficiency. The processing technology is designed and optimized. The membrane preparation efficiency is improved while maintaining a certain separation performance. The polysilsesquioxane/Peek composite membrane has excellent effects for separation and recovery of methanol/toluene and methanol/DMC.

It should be noted that the above embodiments are merely used to illustrate the technical solutions of the present disclosure and are not intended to limit the present disclosure. Although the present disclosure has been described in detail with reference to the preferred embodiments, those of ordinary skill in the art should understand that modifications or equivalent replacements may be made to the technical solutions of the present disclosure without departing from the spirit and scope of the technical solutions of the present disclosure. All such modifications or equivalent replacements shall fall within the scope of the claims.

What is claimed is:

1. A method for preparing a polysilsesquioxane/polyetheretherketone composite membrane, comprising:
obtaining a polysilsesquioxane polymeric sol by mixing an ethanol solvent and a polysilsesquioxane precursor, sequentially adding deionized water and hydrochloric acid after complete dissolution, followed by constant-temperature water bath stirring for performing a polymerization reaction; and
obtaining the polysilsesquioxane/polyetheretherketone composite membrane by coating the polysilsesquioxane polymeric sol on a polyetheretherketone support on which an intermediate layer is formed by knife coating, wherein
a molar ratio of the polysilsesquioxane precursor, the deionized water, and the hydrochloric acid is 1:(6-240):(0.2-1),
a mass fraction of the polysilsesquioxane polymeric sol is 5 wt %-10 wt %; and
the intermediate layer on the polyetheretherketone support is formed by a process including:
subjecting an aqueous phase piperazine solution and an organic phase 1,3,5-benzenetricarbonyl chloride solution to an interfacial polymerization reaction on the polyetheretherketone support, or
subjecting polydimethylsiloxane or amino silicone oil to an oxygen plasma modification on the polyetheretherketone support.

2. The method for preparing the polysilsesquioxane/polyetheretherketone composite membrane according to claim 1, wherein the polysilsesquioxane precursor comprises one of 1,2-bis(triethoxysilyl)ethane, 1,2-bis(triethoxysilyl)ethylene, 1,2-bis(triethoxysilyl)acetylene, 1,2-bis(triethoxyalkyl) methane, or 1,8-bis(triethoxyalkyl)octane.

3. The method for preparing the polysilsesquioxane/polyetheretherketone composite membrane according to claim 1, wherein a stirring temperature of the constant-temperature water bath stirring is 30° C.-80° C., and a stirring time of the constant-temperature water bath stirring is 1 h-5 h.

4. The method for preparing the polysilsesquioxane/polyetheretherketone composite membrane according to claim 1, wherein the subjecting the aqueous phase piperazine solution and the organic phase 1,3,5-benzenetricarbonyl chloride solution to the interfacial polymerization reaction on the polyetheretherketone support comprises:
fixing the polyetheretherketone support, and pouring the aqueous phase piperazine solution onto a surface of the polyetheretherketone support for residence and reaction, and
removing an excess of the aqueous phase piperazine solution on the surface of the polyetheretherketone support with a roller, then pouring the organic phase 1,3,5-benzenetricarbonyl chloride solution for residence to perform the interfacial polymerization reaction to form the intermediate layer, wherein
a concentration of the aqueous phase piperazine solution is 0.5 wt %-2 wt %,
a residence time of the aqueous phase piperazine solution is 1 min-5 min,
a concentration of the organic phase 1,3,5-benzenetricarbonyl chloride solution is 0.01 wt %-0.1 wt %, and
a residence time of the organic phase 1,3,5-benzenetricarbonyl chloride solution is 1 min-5 min; and
a plasma treatment pressure of the oxygen plasma modification is 15 Pa-25 Pa, a treatment power of the oxygen plasma modification is 150 W-250 W, and a treatment time of the oxygen plasma modification is 60 s-180 s.

5. The method for preparing the polysilsesquioxane/polyetheretherketone composite membrane according to claim 1, wherein the coating the polysilsesquioxane polymeric sol on the polyetheretherketone support on which the intermediate layer is formed by the knife coating comprises:
fixing the polyetheretherketone support on which the intermediate layer is formed,
dropping the polysilsesquioxane polymeric sol onto one end of the polyetheretherketone support, and
coating a layer of the polysilsesquioxane polymeric sol with a knife at a constant speed, with a thickness of 200 nm-500 nm, and after calcination and drying, forming the polysilsesquioxane/polyetheretherketone composite membrane.

6. The method for preparing the polysilsesquioxane/polyetheretherketone composite membrane according to claim 5, wherein the constant speed for the coating is 0.5 cm/s-5 cm/s.

7. The method for preparing the polysilsesquioxane/polyetheretherketone composite membrane according to claim 5, wherein a temperature of the calcination and drying is 50° C.-200° C., and a time of the calcination and drying is 15 min-60 min.

8. A polysilsesquioxane/polyetheretherketone composite membrane, which is prepared by the method according to claim 1, wherein the polysilsesquioxane/polyetheretherketone composite membrane comprises a support, the intermediate layer, and a polymeric sol layer, wherein
a material of the support is polyetheretherketone, and a pore size of the support is 1 nm-100 nm,
a material of the polymeric sol layer is the polysilsesquioxane polymeric sol,
the intermediate layer is formed on the support, and
the polymeric sol layer is coated on a surface of the intermediate layer.

9. A use of the polysilsesquioxane/polyetheretherketone composite membrane prepared by the method according to claim 1 in separating an organic mixed solvent, comprising:
separating the organic mixed solvent by pervaporation using the polysilsesquioxane/polyetheretherketone composite membrane, wherein
a separation system of the organic mixed solvent comprises methanol/toluene and methanol/dimethyl carbonate.
